(19) United States
(12) Patent Application Publication
(10) Pub. No.: US 2026/0069220 A1
(43) Pub. Date: Mar. 12, 2026

(54) PRIORITIZATION AND AGGREGATION SCHEME OF ALARMS FOR PATIENT MONITORING SYSTEM

(71) Applicant: Draeger Medical Systems, Inc., Andover, MA (US)

(72) Inventors: Ketaki Adwait Muthal, Winchester, MA (US); Alyssa Valentin, Andover, MA (US)

(21) Appl. No.: 18/937,918

(22) Filed: Nov. 5, 2024

Related U.S. Application Data
(60) Provisional application No. 63/693,038, filed on Sep. 10, 2024.

(51) Int. Cl. A61B 5/00; G08B 21/18; G08B 25/14

(52) U.S. Cl. CPC A61B 5/746; A61B 5/7264; A61B 5/742; G08B 21/182; G08B 25/14

(57) ABSTRACT

In a central monitoring solution, one or more predetermined criteria are established which includes data from several clinical parameters to provide visual indications of patient alarm priority and identify patients that may need more attention than others. If preset criteria for appropriate zones are met, the patient will be indicated either in “red zone” demanding immediate attention; or in a “yellow zone” indicating moderate level of attention; or in a “blue zone”. Indicating no criteria are met. This color-coding scheme will help care givers/teletechs an opportunity to sort patients based on their clinical condition.

### CROSS REFERENCE TO RELATED APPLICATIONS

The present application claims priority to and the benefit of U.S. Prov. Pat. App. Ser. No. 63/693,038, which was filed on Sep. 10, 2024, for all purposes, including the right of priority, which application is hereby incorporated herein by reference in its entirety and to the extent that is not inconsistent with the present disclosure.

### BACKGROUND

The present disclosure relates generally to the field of medical patient monitoring. More particularly, the present disclosure relates to filtering, prioritizing, and aggregation of patient alarms generated in patient monitoring systems.

### SUMMARY

The method and apparatus of the present disclosure may be implemented on a central monitoring station in cooperation with remote (e.g., bedside) patient monitors, and may follow one or more predetermined criteria that include/includes data from several clinical parameters to indicate a Patient Alarm Prioritization (“PAP”) scheme for patients that need more attention than others. In some embodiments described herein, the PAP scheme is a traffic lighting scheme. If preset criteria for appropriate zones are met, the patient will be indicated either in “red zone” demanding immediate attention; or in “yellow zone” indicating moderate level of attention; or in “blue zone” indicating no criteria are met. This color-coding scheme will help care givers/teletechs an opportunity to sort patients based on their clinical condition.

In a first aspect, then, a computer-implemented method of prioritizing patient alarms is deployed in a patient monitoring system including a central monitoring station. The computer-implemented method comprises: receiving a patient alarm issued by the patient monitoring system when a monitored parameter deviates from a predetermined value; tracking a total number of patient alarms for a set of predefined parameters in a predefined time period; defining a plurality of priority levels for patient alarms, each priority level defined by a threshold number of patient alarms; and for each patient being monitored, displaying a visual indicator at the central monitoring station when the total number of patient alarms exceeds the threshold number of patient alarms for the set of predefined parameters in each of the plurality of priority levels, the visual indicator identifying the priority level reached by the monitored patient.

In a second aspect, a central monitoring station for deployment in a clinical care environment comprises one or more processors, a display, and a memory. On the memory reside instructions that, when executed by the one or more processors, cause the one or more processors to perform a method. The method comprises: receiving a patient alarm issued by the patient monitoring system when a monitored parameter deviates from a predetermined value; tracking a total number of patient alarms for a set of predefined parameters in a predefined time period; defining a plurality of priority levels for patient alarms, each priority level defined by a threshold number of patient alarms; and for each patient being monitored, displaying a visual indicator on the display when the total number of patient alarms exceeds the threshold number of patient alarms for the set of predefined parameters in each of the plurality of priority levels, the visual indicator identifying the priority level reached by the monitored patient.

In a third aspect, a patient monitoring system is deployed in a clinical care environment. The patient monitoring system comprises a plurality of medical devices and a central monitoring station. The medical devices monitor a respective physiological condition for each of a plurality of patients. The monitoring includes monitoring one or more physiological parameters and issuing one or more alarms when a monitored parameter deviates from a predetermined value. The central monitoring station performs a method comprising: receiving the patient alarms issued by the plurality of medical devices; tracking a total number of patient alarms for a set of predefined parameters in a predefined time period; defining a plurality of priority levels for the patient alarms, each priority level defined by a threshold number of patient alarms; and for each patient being monitored, displaying a visual indicator at the central monitoring station when the total number of patient alarms exceeds the threshold number of patient alarms for the set of predefined parameters in each of the plurality of priority levels, the visual indicator identifying the priority level reached by the monitored patient.

The above presents a simplified summary of the invention in order to provide a basic understanding of some aspects of the invention. This summary is not an exhaustive overview of the invention. It is not intended to identify key or critical elements of the invention or to delineate the scope of the invention. Its sole purpose is to present some concepts in a simplified form as a prelude to the more detailed description that is discussed later.

### BRIEF DESCRIPTION OF THE DRAWINGS

In the drawings, like reference numbers generally indicate identical, functionally similar, and/or structurally similar elements.

FIGS. 1A, 1B, and 1C are a flow diagram illustrating an alert sequence for a first level of prioritization of patient alarms according to one or more examples;

FIGS. 2A, 2B, and 2C are a flow diagram illustrating an alert sequence for a second level of prioritization of patient alarms according to one or more examples;

FIGS. 3A, 3B, and 3C are a flow diagram illustrating an alert sequence for a third level of prioritization of patient alarms according to one or more examples;

FIG. 4 is a representation of a user interface for a patient alarm prioritization system incorporated into a patient monitoring system according to one or more examples;

FIG. 5 is a further representation of a user interface for a patient alarm prioritization system incorporated into a patient monitoring system according to one or more examples;

FIG. 6 is a further representation of a user interface for a patient alarm prioritization system incorporated into a patient monitoring system according to one or more examples; and

FIG. 7 is a further representation of a user interface for a patient alarm prioritization system incorporated into a patient monitoring system according to one or more examples.

FIG. 8 depicts a scenario in which a patient monitoring system may be implemented according to one or more examples.

FIG. 9A, FIG. 9B are block diagrams of selected aspects of the medical device and the central monitoring system, respectively, first shown in FIG. 8.

FIG. 10 depicts a scenario in which the patient monitoring system of FIG. 8 may be implemented according to one or more examples.

While the disclosed technique is susceptible to various modifications and alternative forms, the drawings illustrate specific embodiments herein described in detail by way of example. It should be understood, however, that the description herein of specific embodiments is not intended to limit that which is claimed to the particular forms disclosed, but on the contrary, the intention is to cover all modifications, equivalents, and alternatives falling within the spirit and scope of the appended claims.

### DETAILED DESCRIPTION

Illustrative examples of the subject matter claimed below are disclosed. In the interest of clarity, not all features of an actual implementation are described for every example in this specification. It will be appreciated that in the development of any such actual implementation, numerous implementation-specific decisions may be made to achieve the developers' specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

The expressions such as “include” and “may include” which may be used in the present disclosure denote the presence of the disclosed functions, operations, and constituent elements, and do not limit the presence of one or more additional functions, operations, and constituent elements. In the present disclosure, terms such as “include” and/or “have”, may be construed to denote a certain characteristic, number, operation, constituent element, component or a combination thereof, but should not be construed to exclude the existence of or a possibility of the addition of one or more other characteristics, numbers, operations, constituent elements, components or combinations thereof.

As used herein, the article “a” is intended to have its ordinary meaning in the patent arts, namely “one or more.” Herein, the term “about” when applied to a value generally means within the tolerance range of the equipment used to produce the value, or in some examples, means plus or minus 10%, or plus or minus 5%, or plus or minus 1%, unless otherwise expressly specified. Further, herein the term “substantially” as used herein means a majority, or almost all, or all, or an amount with a range of about 51% to about 100%, for example. Moreover, examples herein are intended to be illustrative only and are presented for discussion purposes and not by way of limitation.

As used herein, to “provide” an item means to have possession of and/or control over the item. This may include, for example, forming (or assembling) some or all of the item from its constituent materials and/or, obtaining possession of and/or control over an already-formed item.

Unless otherwise defined, all terms including technical and/or scientific terms used herein have the same meaning as commonly understood by one of ordinary skill in the art to which the present disclosure pertains. In addition, unless otherwise defined, all terms defined in generally used dictionaries may not be overly interpreted. In the following, details are set forth to provide a more thorough explanation of the embodiments. However, it will be apparent to those skilled in the art that embodiments may be practiced without these specific details. In other instances, well-known structures and devices are shown in block diagram form or in a schematic view rather than in detail in order to avoid obscuring the embodiments. In addition, features of the different embodiments described hereinafter may be combined with each other, unless specifically noted otherwise. For example, variations or modifications described with respect to one of the embodiments may also be applicable to other embodiments unless noted to the contrary.

Further, equivalent or like elements or elements with equivalent or like functionality are denoted in the following description with equivalent or like reference numerals. As the same or functionally equivalent elements are given the same reference numbers in the figures, a repeated description for elements provided with the same reference numbers may be omitted. Hence, descriptions provided for elements having the same or like reference numbers are mutually exchangeable.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

In the present disclosure, expressions including ordinal numbers, such as “first”, “second”, and/or the like, may modify various elements. However, such elements are not limited by the above expressions. For example, the above expressions do not limit the sequence and/or importance of the elements. The above expressions are used merely for the purpose of distinguishing an element from the other elements. For example, a first box and a second box indicate different boxes, although both are boxes. For further example, a first element could be termed a second element, and similarly, a second element could also be termed a first element without departing from the scope of the present disclosure.

A sensor refers to a component which converts a physical quantity to be measured to an electric signal, for example, a current signal or a voltage signal. The physical quantity may for example comprise electromagnetic radiation (e.g., photons of infrared or visible light), a magnetic field, an electric field, a pressure, a force, a temperature, a current, or a voltage, but is not limited thereto.

Various examples of a Patient Alarm Prioritization system (“PAP system”) described herein assist caregivers with patient prioritization and alarm aggregation. Caregivers are experiencing an increase in workload, patient-to-caregiver ratios, acuity of patients, number of clinical alarms, and caregiver fatigue, all of which may have negative impacts on patient outcomes.

In an environment where caregivers are caring for a higher number and acuity of patients as well as responding to more alarms, it is not only the management of the alarms themselves that becomes important in decreasing caregiver fatigue, but also assistance with patient prioritization in response to the alarms. With each patient alarming 150 to 400 times or more per shift (multiplied by the number of patients the caregiver is caring for), assisting them in prioritizing which patients they must provide attention to first is imperative, as the caregivers are responding to hundreds of alarms for each patient each shift.

While it is not always possible to minimize/eliminate alarms due to their significance, assisting the caregiver in interpreting patient priority based on alarms helps them in determining which patients require attention first. This is one intended function of the PAP system as described herein. In one or more examples, the PAP system provides patient-specific visual indicators via the central station, which identify patients on a low/medium/high priority scale based on which alarms have occurred. The caregiver is able to select the alarms that are most significant for each patient, select a time frame, and select the number of these alarms that must occur in that time frame in order to trigger the activation of the low/medium/high priority visual indicators (e.g., differentiated by color). When a PAP indicator is triggered, it will appear next to the respective patient's name on the central station display as a small blinking visual indicator. When the caregiver clicks on the indicator, this will open a historical list of alarms that met PAP criteria, transitioning the visual indicator from a blinking state to a static state. The caregiver also has the option to reset the PAP indicator from this screen which clears the indicator from the display as well as the historical list of applicable alarms, thus resetting the process.

As the caregiver may be responding to many alarms for many different patients at any given time, these patient-specific indicators visually present which patients have met pre-selected PAP criteria, providing an overview of patient prioritization and helping the caregiver determine which patients may need closer care/monitoring. This may also help caregivers in assessing which patients may be deteriorating, which patients may need to be evaluated for increased levels of care, which patients may not be suitable for discharge, etc. This also helps the caregiver in understanding which patients may be alarming more frequently than they had been previously, aiding in recognition of potential patient deterioration and increased acuity.

In addition, the feature also provides machine learning/AI generated alarm setting suggestions based on the alarms that have occurred for each patient. As the feature monitors the frequency of occurrence of selected alarms, it is able to suggest changes to alarm settings that may better suit each patient and decrease caregiver alarm fatigue.

Further, the feature also provides the ability to organize the view of patients at the central station based on PAP prioritization. When PAP is enabled on a central station, users have a PAP view option which organizes the patients on the central station screen based on their PAP priority indicators, placing patients who have met criteria to trigger high priority (RED) indicators at the top of the screen, followed by medium priority (YELLOW) indicator patients, low priority (BLUE) indicator patients, and finally patients who have not met criteria to trigger the PAP indicator as you move downwards. Not only useful for on-unit caregivers, this feature and PAP view option is also very beneficial for off-unit caregivers, such as telemetry technicians who monitor more than 40 patients at once in war-room environments.

Although various examples are described herein wherein the PAP system visualization is color-coded, it is to be understood that in other examples, different visualization methodologies may be implemented. For example, in addition to or instead of color coding, the visualizations could be alphanumeric, symbolic, or some combination thereof.

The following Table 1 is an example of an implementation of a patient alarm filtering, prioritization, and aggregation process according to one or more embodiments.

TABLE 1

| | SELECT | | |
|---|---|---|---|
| SELECT | ALARM | SELECT | |
| TIME | LEVEL | ALARM TYPE | CONDITION |
| 5/10/15 | High | Select up to N | If predetermined number of |
| minutes | only | arrhythmia and | discrete arrhythmia or parameter |
| | | N parameter | alarms are recorded, then |
| | | alarms | indicate with RED |
| 5/10/15 | High + | Select up to N | If predetermined number of |
| minutes | Medium | arrhythmia and | discrete arrhythmia or parameter |
| | | N parameter | alarms are recorded, then |
| | | alarms | indicate with YELLOW |
| 5/10/15 | Medium | Select up to N | If predetermined number of |
| minutes | only | arrhythmia and | discrete arrhythmia or parameter |
| | | N parameter | alarms are recorded, then |
| | | alarms | indicate with BLUE |

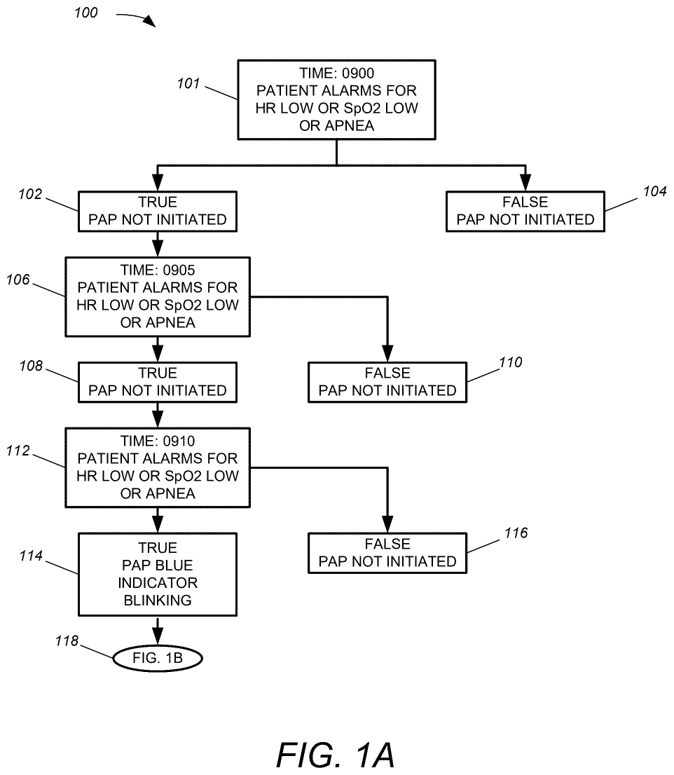

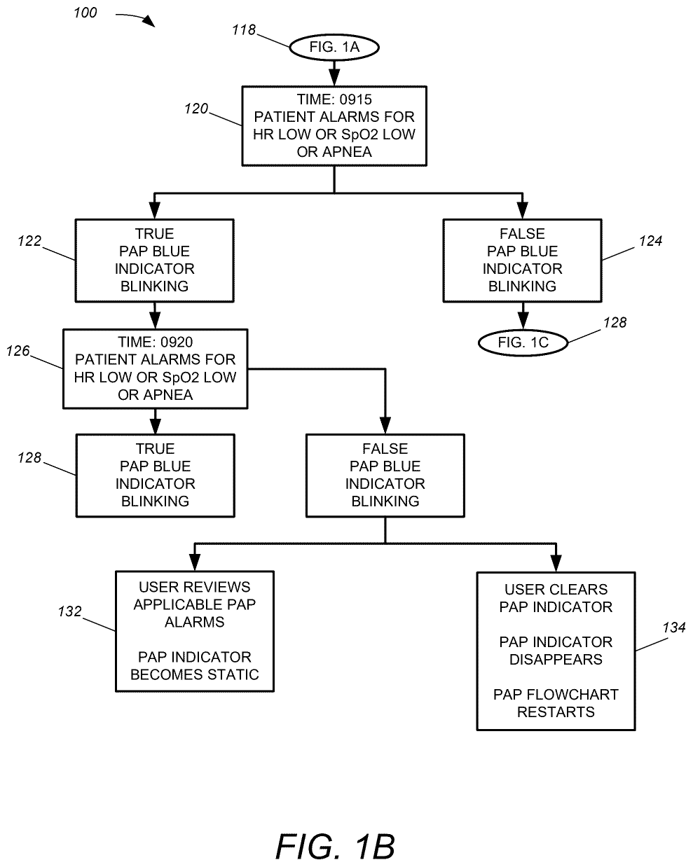

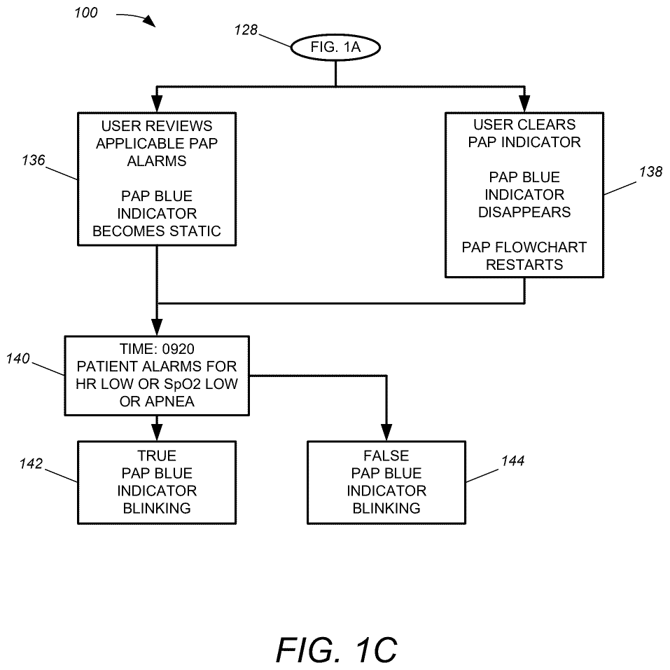

Referring now to FIGS. 1A, 1B, and 1C, 2A, 2B, and 2C, and 3A, 3B, and 3C, there are shown a flow diagrams 100, 200, and 300, respectively, illustrating an alert sequence for a multi-tiers prioritization of patient alarms according to one or more examples. In the example represented by flow diagrams 100, 200, and 300, the PAP system is enabled with respect to heart rate (HR), blood oxygenation (Sp02) and apnea sensing parameters for the patients. In this example, the threshold number of alarms for triggering a “BLUE” indication to the caregiver (flow diagram 100) is set to three (3), the threshold number of alarms for triggering a “YELLOW” indication to the caregiver (flow diagram 200) is set to six (6), and the threshold number of alarms for triggering a “RED” indication to the caregiver (flow diagram 300) is set to nine (9).

As shown in FIGS. 1A, 1B, and 1C, in block 101, at time 0900, the patient has alarmed for one of the specified parameters (HR, SpO2, or apnea). If this is determined to be a false alarm, then in block 104, the PAP system is not initiated. Likewise, in block 102, if the alarm is valid ‘true”), the PAP system is not initiated, since as noted above the threshold for initiation at the lowest (BLUE) alarm level is three alarms. With continued reference to FIG. 1A, in block 106, at time 0905, the patient has alarmed for one of the specified parameters. If this alarm is determined to be false, then in block 110, the PAP system is not initiated. If the alarm is determined to be valid (“true”), in block 108 the PAP system again is not initiated, because the threshold for initiation at this level is three alarms.

In block 112, the patient has alarmed for one of the specified parameters. If this alarm is determined to be false, then in block 116, the PAP system is not initiated. If the alarm is determined to be valid (“true”), in block 114 the PAP system is initiated, with the BLUE indicator blinking on the central monitor, because the threshold for initiation at this level is three alarms, and the alarm at block 112 was the third in this example.

Connector block 118 shows the transition from FIG. 1A to FIG. 1B. Referring now to FIG. 1B, in block 120, at time 0915, the patient alarms for one of the specified parameters. If this alarm is determined to be false, then in block 124, the PAP system continues to maintain the blinking BLUE indicator. Likewise, if the alarm in block 120 is valid (“true”), then in block 122 the PAP system continues to maintain the blinking BLUE indicator, since the threshold for escalation to a higher priority level is six alarms, whereas the alarm at block 120 is only the fourth in this example.

In block 126, at time 0920, the patient alarms for one of the specified parameters. If this alarm is determined to be false, then in block 130, the PAP system continues to maintain the blinking BLUE indicator. Likewise, in block 128, if the alarm is at block 126 is determined to be valid (“true”), PAP system continues to maintain the blinking blue indicator, since the threshold for escalation to a higher priority level is six alarms, whereas the alarm at block 120 is only the fifth in this example.

Following the false alarm in block 130, in block 132, the user reviews the applicable PAP alarms. This causes the BLUE PAP indicator to become static, reflecting the user's attention to the alarm status. Alternatively, in block 134, the user may determine that the patient status is acceptable and may clear the PAP indicator. This causes the PAP indicator to disappear, and operation of the PAP system restarts.

Connector block 128 shows the transition from FIG. 1B to FIG. 1C, between block 124 (BLUE indicator blinking) to blocks 136 and 138. In block 136, the user may review the applicable PAP alarms, causing the PAP BLUE indicator to transition to static (on). Alternatively, in block 138, the user may clear the PAP indicators, causing the PAP indicator to disappear, and restarting the PAP system.

With continued reference to FIG. 1C, in block 140, the patient alarms for one of the specified parameters. This is the fifth alarm in this example. If the PAP alarm is determined to be false, then in block 144, the PAP BLUE indicator remains static. However, if the alarm in block 140 is determined to be true, then in block 142, the PAP BLUE indicator reverts to blinking.

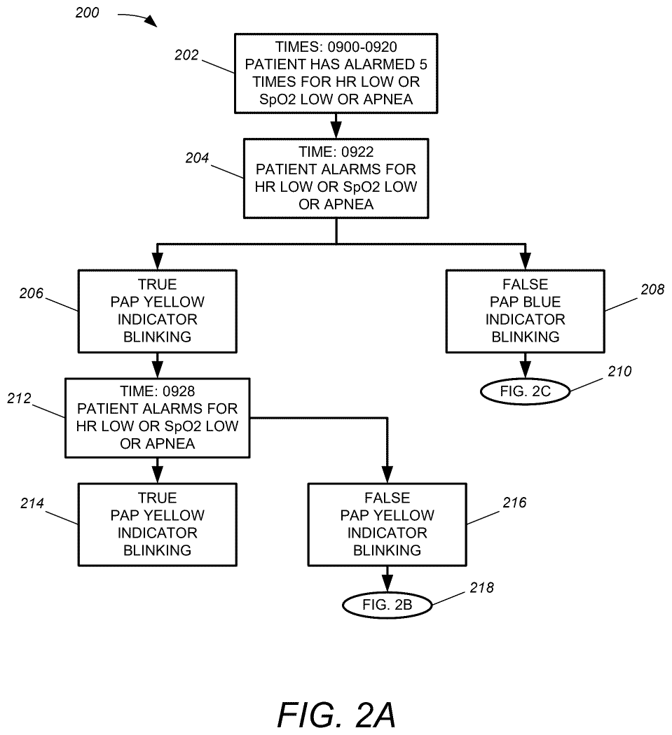

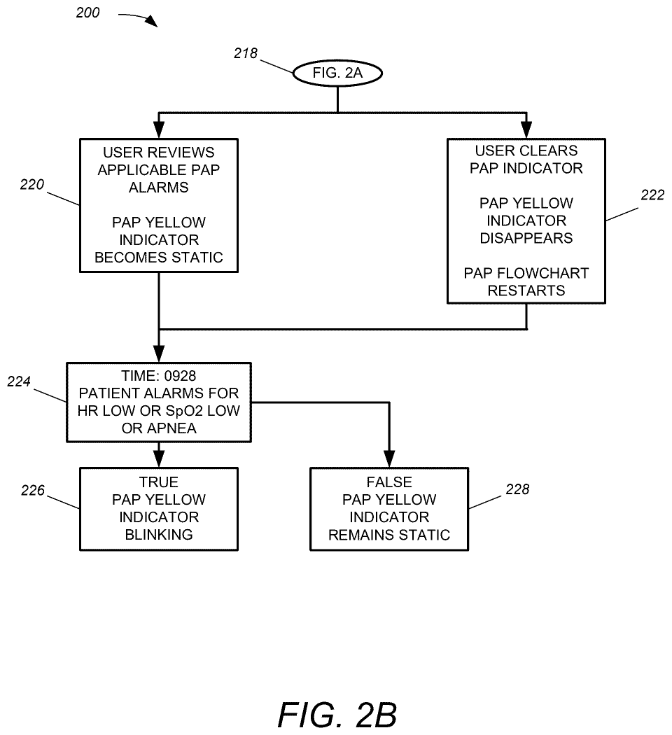

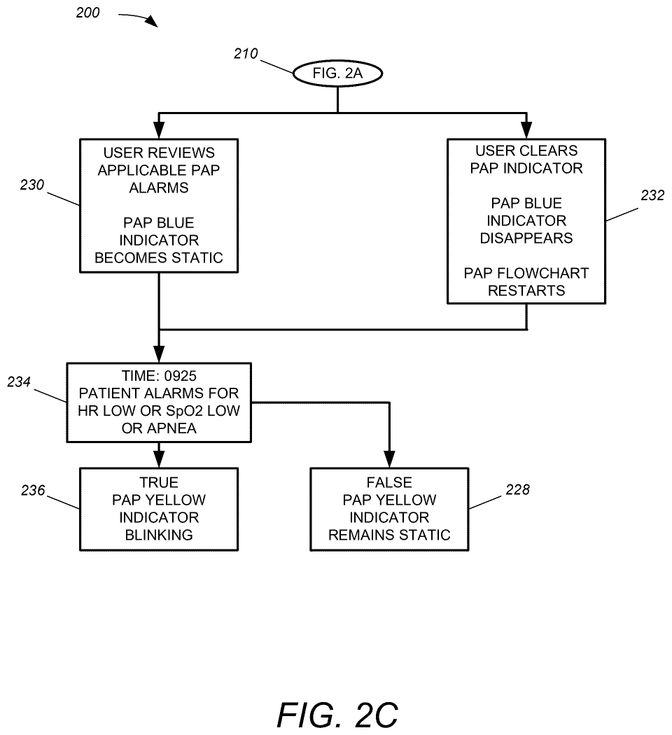

Referring next to FIGS. 2A, 2B, and 2C, there is shown a flow diagram 200 illustrating an alert sequence for a second level of prioritization of patient alarms according to one or more examples. As noted at block 202 in FIG. 2A, between times 0900 and 0920, the patient has alarmed five times for the specified parameters.

At block 204 in FIG. 2A, at time 0922, the patient alarms for one of the specified parameters. This represents the sixth alarm for the patient in this example, thus entering the second (YELLOW) priority level of the PAP system. In block 208, if the alarm is determined to be false, the PAP system maintains the blinking BLUE PAP indicator. However, in block 206, if the alarm is determined to be true, the PAP system will initiate a blinking YELLOW indicator.

With continued reference to FIG. 2A, in block 212, at time 0928, the patient alarms for one of the specified parameters. This is the seventh alarm in this example. If the patient alarm in block 212 is determined to be false, then in block 216, the PAP system maintains the blinking YELLOW indicator set in block 206. Likewise, in block 214, if the patient alarm in block 212 is determined to be true, then the PAP system maintains the blinking YELLOW indicator, since the threshold for transitioning to the next higher indicator level (BLUE) is nine alarms.

Connector block 218 shows the transition from FIG. 2A to FIG. 2B, between block 216 (YELLOW indicator blinking) to blocks 220 and 222. In block 222, the user may clear the PAP indicators, causing the YELLOW indicator to disappear and restarting the PAP system. Alternatively, in block 220, the user may review the applicable PAP alarms, causing the PAP YELLOW indicator to become static.

In block 224 in this example, at time 0928, the patient alarms for one of the specified parameters. This is the eighth alarm in the illustrated example. As shown in block 228, if this alarm is determined to be false, the PAP system maintains the static YELLOW indicator. However, as shown in block 226, if the alarm in block 224 is determined to be true, then the PAP system reverts to a blinking YELLOW PAP indicator.

Connector block 210 shows the transition from FIG. 2A to FIG. 2C, between block 208 (BLUE indicator blinking) to blocks 230 and 232. In block 230, the user may review applicable PAP alarms, causing the BLUE indicator to become static. Alternatively, in block 232, the user may clear the PAP indicators, causing the BLUE indicator to disappear and restarting the PAP process.

In block 234 in FIG. 2C, at time 0925, the patient alarms for one of the specified parameters. This is the seventh alarm in the illustrated example. If the alarm in block 234 is determined to be false, then in block 238, the PAP system maintains the static BLUE indicator. On the other hand, in block 236, if the alarm in block 234 is determined to be true, then the PAP system triggers a blinking YELLOW indicator.

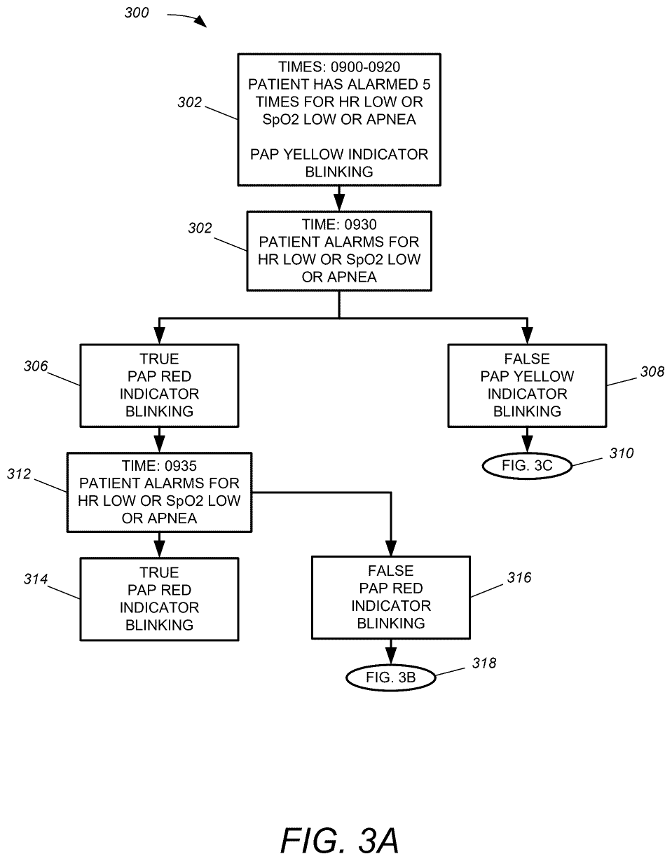

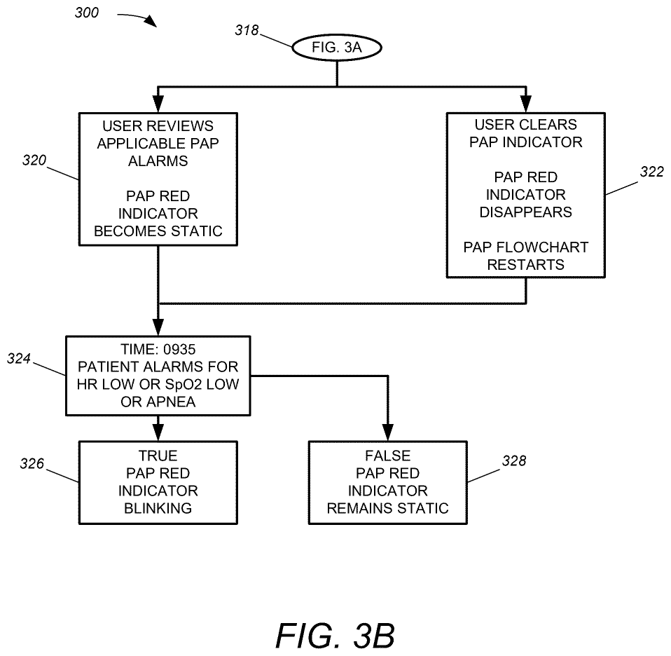

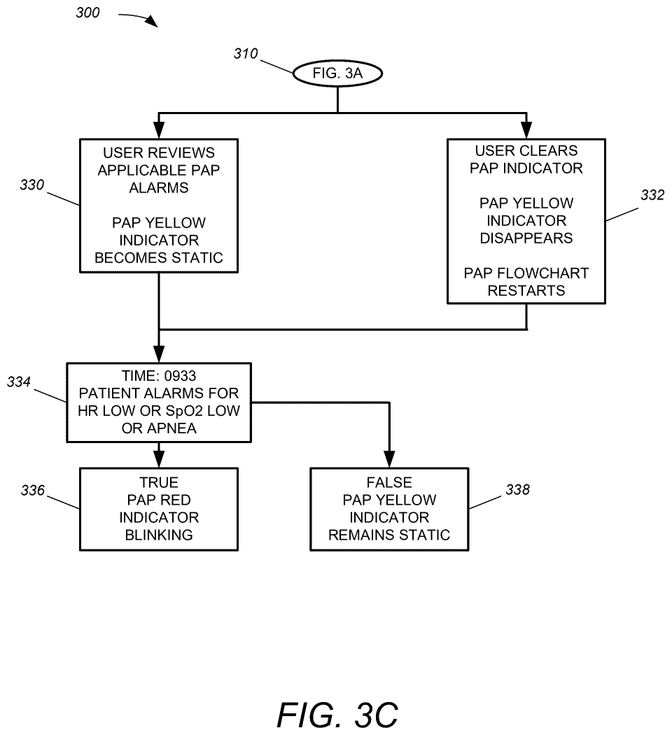

Referring next to FIGS. 3A, 3B, and 3C, there is shown a flow diagram 300 illustrating an alert sequence for a third level of prioritization of patient alarms according to one or more examples. As noted at block 302 in FIG. 3A, between times 0900 and 0928 (block 224 in FIG. 2B), the patient has alarmed eight times for the specified parameters. Then, in block 304 in FIG. 3A, at time 0930, the patient alarms for one of the specified parameters. This is the ninth alarm in the illustrated example. If the alarm in block 304 is determined to be false, then in block 308, the PAP system maintains the static YELLOW indicator. On the other hand, if the alarm in block 304 is determined to be true, then in block 306, the PAP system triggers a blinking RED indicator.

With continued reference to FIG. 3A, in block 312, at time 0935, the patient alarms for one of the specified parameters. This is the tenth alarm in the illustrated example. If the alarm in block 312 is determined to be false, then in block 316, the PAP system maintains the blinking RED indicator. likewise, if the alarm in block 312 is determined to be true, then in block 314, the PAP system maintains the blinking RED indicator.

Connector block 318 shows the transition from FIG. 3A to FIG. 3B, between block 316 (RED indicator blinking) to blocks 320 and 322. In block 320, the user may review applicable PAP alarms, causing the PAP system to make the RED indicator static. In block 322, the user may clear the PAP indicators, causing the RED indicator to disappear and restarting the PAP process.

In block 324 in FIG. 3B, at time 0935, the patient alarms for one of the specified parameters. This is the eleventh alarm in the illustrated example. If the alarm in block 324 is determined to be false, then in block 328, the RED indicator remains static. On the other hand, as shown at block 326, if the alarm in block 324 is determined to be true, then the PAP system triggers a blinking RED indicator.

Connector block 310 shows the transition from FIG. 3A to FIG. 3C, between block 308 (YELLOW indicator blinking) to blocks 330 and 332. In block 330, the user may review applicable PAP alarms, causing the PAP system to make the YELLOW indicator static. In block 332, the user may clear the PAP indicators, causing the YELLOW indicator to disappear and restarting the PAP process.

In block 334 in FIG. 3C, at time 0933, the patient alarms for one of the specified parameters. This is a tenth alarm in the illustrated example. If the alarm in block 334 is determined to be false, then in block 338, the PAP system causes the YELLOW indicator to remain static. On the other hand, as shown at block 336, if the alarm in block 334 is determined to be true, then the PAP system triggers a blinking RED indicator.

It is contemplated that the parameters used in the PAP system may be user configurable. It is contemplated that the parameters used in the PAP system may be customizable for a given care area, patient population, disease state, known medication, etc. It is contemplated that the parameters used in the PAP system may be selectable from a list comprising: heart rate (HR), blood oxygenation (Sp02), apnea sensing, temperature, number of total arrythmias, specific types of arrythmias, ECG, respiration rate, moisture sensing, and various other physiological parameters.

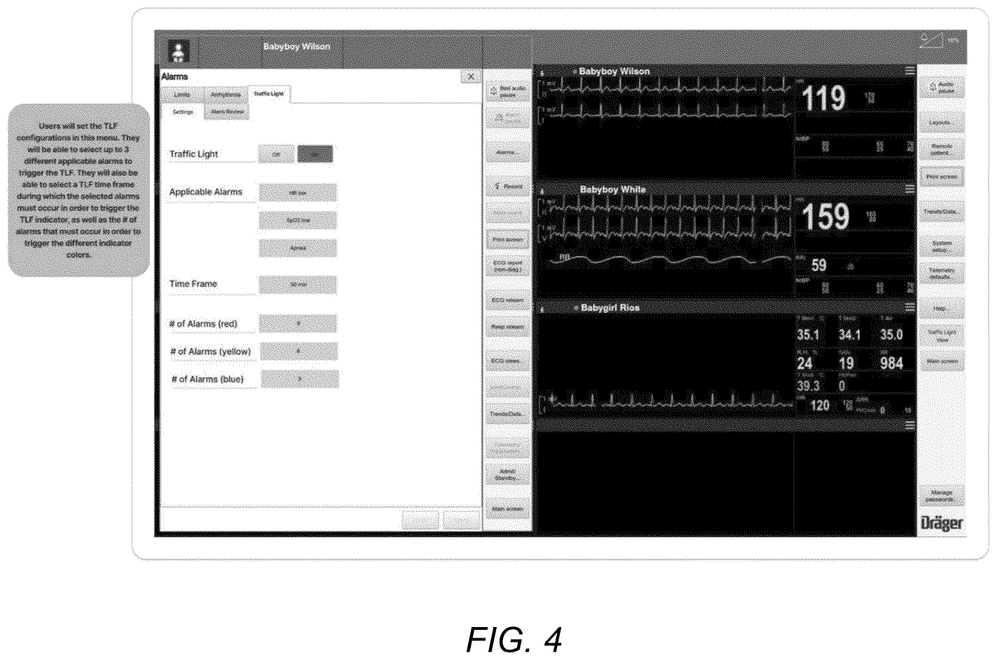

FIG. 4 is a representation of a user interface for a patient alarm prioritization system incorporated into a patient monitoring system according to one or more examples. FIG. 4 in particular depicts the user interface by which a user (caregiver) may set PAP system parameters. In the example of FIG. 4, the user may select up to three different applicable alarms to trigger the PAP system. The user may also select a PAP time frame during which the selected alarms must occur in order to trigger the PAP system indicator(s), as well as the number of alarms that must occur in order to trigger the different indicators (BLUE, YELLOW, RED).

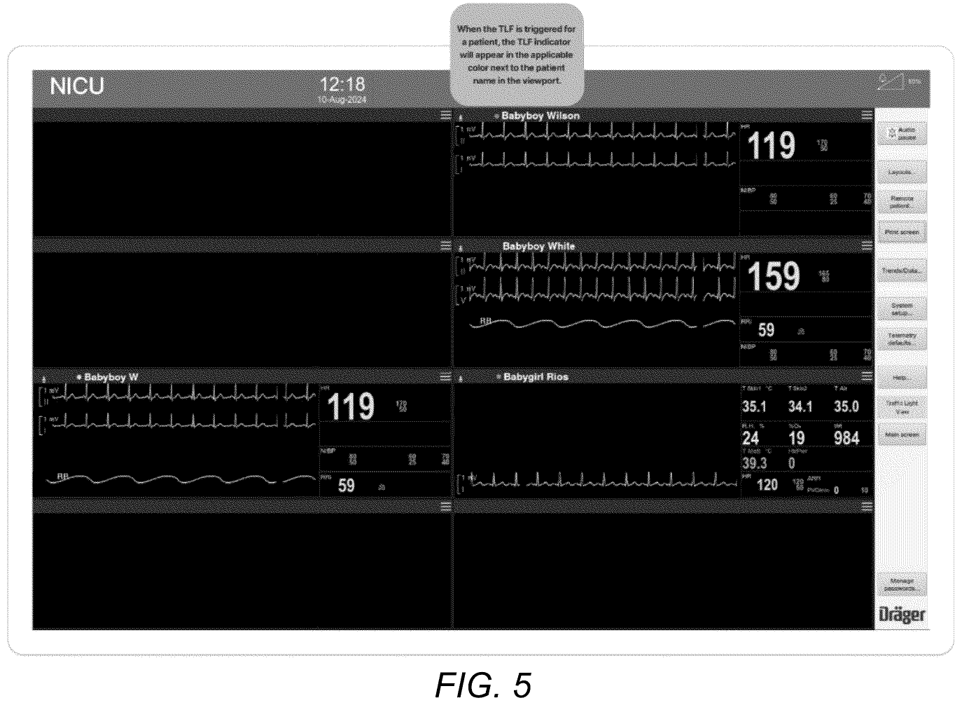

As shown in FIG. 5, when the PAP system is triggered for a patient, the PAP indicator will appear in the applicable color next to the patient name in the viewport.

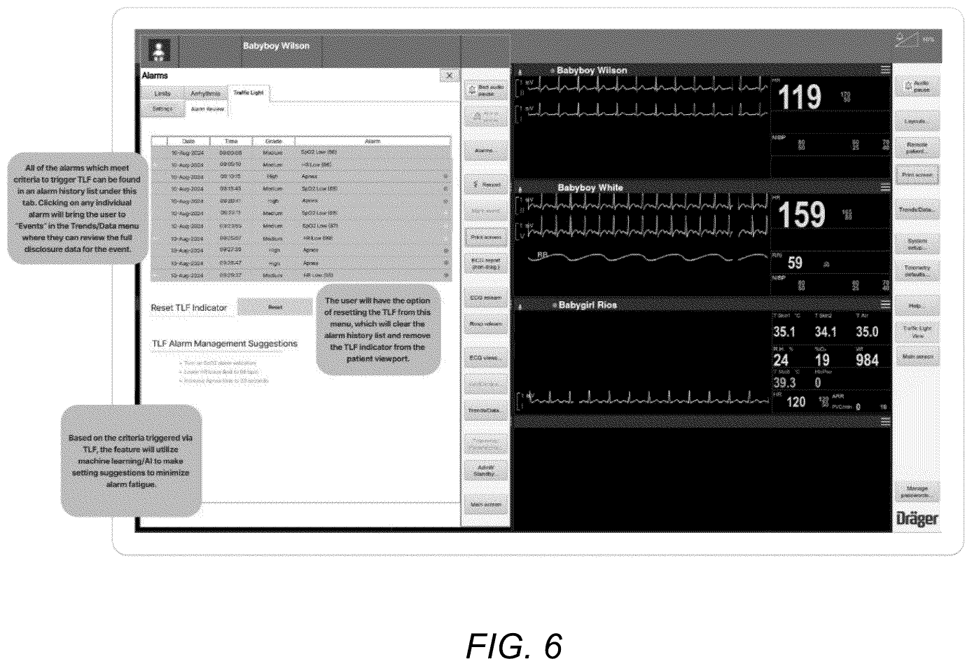

As shown in FIG. 6, all of the alarms that meet the criteria to trigger the PAP system can be found in an alarm history list associated with each respective patient. Selecting any individual alarm will enable the user to view an “Events” history, where the user can review the full data associated with the events. The user may have an option of resetting the PAP system from the menu shown in FIG. 6, which will clear the alarm history list and remove the PAP indicator from the patient viewport. Further, based on the criteria triggered via the PAP system, a machine learning algorithm may be utilized to provide suggestions to the user for settings adjustments, potentially leading to even further lessened alarm fatigue. The machine learning algorithm inputs may include patient parameters selected, specific time thresholds used, and patient outcomes.

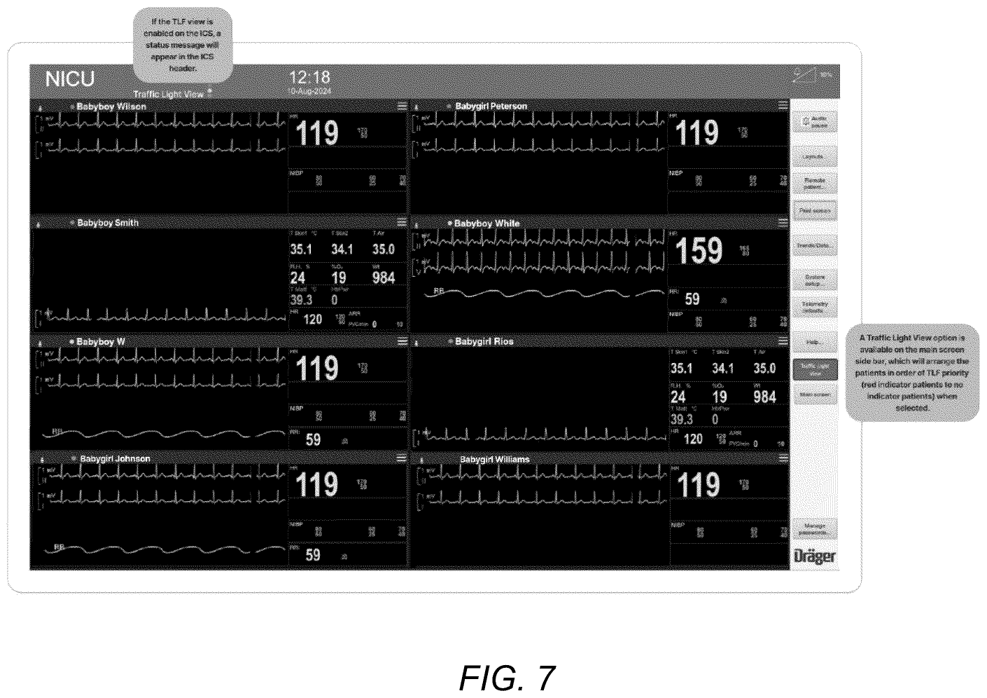

As shown in FIG. 7, if the PAP system view is enabled for a particular patient, a status message may be displayed at a central monitoring station (“ICS”). A PAP system “view” option may be available at the ICS which may arrange the patients in order of PAP system priority.

A computing resource implementing a method of patient alarm prioritization may be provided. The computing resource may be, for example and without limitation, a personal desktop computer (e.g., a personal computer), a mobile computing platform (e.g., a laptop or tablet), or a desktop computer accessing cloud computing resources. The computing resource may include at least one hardware processor and a non-transitory machine-readable storage medium. The machine-readable medium may store instructions, that when executed by the hardware processor (either directly or via emulation/virtualization), cause the hardware processor to perform the method of patient alarm prioritization described above.

In various examples, the hardware processor may be, for example and without limitation, a microcontroller, a central processing unit (“CPU”), a digital signal processor (“DSP”), a programmed logic array (“PLA”), or a custom processing circuit. Instructions may be executed by one or more processors, such as one or more central processing units (“CPU”), digital signal processors (“DSPs)”, general purpose microprocessors, application specific integrated circuits (“ASICs”), field programmable logic arrays (“FPGAs”), or other equivalent integrated or discrete logic circuitry. Accordingly, the term “processor,” as used herein refers to any of the foregoing structure or any other structure suitable for implementation of the techniques described herein. In addition, in some aspects, the functionality described herein may be provided within dedicated hardware and/or software modules. Also, the techniques could be fully implemented in one or more circuits or logic elements. A “controller,” including one or more processors, may use electrical signals and digital algorithms to perform its receptive, analytic, and control functions, which may further include corrective functions. Thus, a controller is a specific type of processing circuitry, comprising one or more processors and memory, that implements control functions by way of generating control signals.

A computer-readable media may be any available media that may be accessed by a computer. By way of example, such computer-readable media may comprise random access memory (“RAM”), read-only memory (“ROM”), electrically-erasable/programmable read-only memory (“EEPROM”), compact disc ROM (“CD-ROM”) or other optical disk storage, magnetic disk storage or other magnetic storage devices, or any other medium that may be used to carry or store desired program code in the form of instructions or data structures and that may be accessed by a computer. Disk and disc, as used herein, includes compact disc (“CD”), laser disc, optical disc, digital versatile disc (“DVD”), floppy disk and Blu-ray® disc where disks usually reproduce data magnetically, while discs reproduce data optically with lasers.

Note also that the software implemented aspects of the subject matter hereof are usually encoded on some form of program storage medium or implemented over some type of transmission medium. The program storage medium is a non-transitory medium and may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The claimed subject matter is not limited by these aspects of any given implementation.

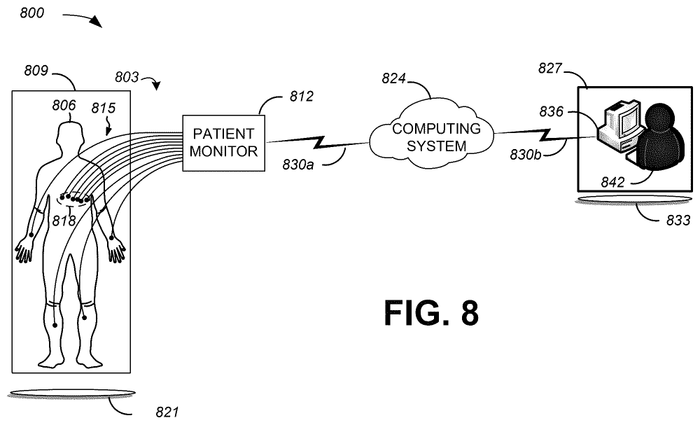

FIG. 8 depicts a clinical care environment 800 in which patient monitoring system 803 may be implemented according to one or more examples. A patient 806 in a bed or pallet 809 physically interfaces with a medical device 812. Those in the art will appreciate that care may be delivered to a patient in a typical healthcare environment using a multiplicity of devices that might generate alarms. These alarms may be associated with the purpose and operation of the respective medical device and, so, may differ from those alarms issued by other devices.

Although not shown, those ordinarily skilled in the art having the benefit of this disclosure will appreciate that the medical monitoring system 803 will typically include a wide variety and number of other medical and network devices. Such other medical devices may include, for example and without limitation, IV drips, therapy devices, isolettes, movement detectors, etc. Other network devices may include, without limitation, gateways, antennas, servers, routers, and various other computing devices. These other medical and network devices work together and individually to impart functionality of the network 115 as described herein.

Accordingly, the medical device 812 in the clinical care environment 800 is representative of a suite of medical devices that might be encountered in a typical healthcare environment. All behaviors and functionalities relevant to the claimed subject matter can be extrapolated to other medical devices that might be present but not shown. It is nevertheless to be understood that the issuance of alarms and the types of those alarms may vary depending on the medical device that issues the alarm.

The medical device 812 in the clinical care environment 800 is a patient monitor that is monitoring one or more physiological parameters of the patient 806 but may be some other type of medical device in other embodiments. The physiological parameters are monitored by the medical device 812 over a plurality of leads 815 including sensors 818. The conductive leads 815 are affixed to the patient 806 disposed upon a bed or pallet 809 and data is being acquired. The patient 806 is located at what may be referred to as a “local” location 821, which is “local” because it is where the patient 806 is located.

Those in the art having the benefit of this disclosure will appreciate that a typical healthcare environment might monitor for a variety of physiological parameters such a heart rate, respiration rate, blood oxygen content, etc. In general terms, a patient monitor acquires data, processes and/or analyzes such acquired data. The processing and analysis might result in the detection of an alarm condition and, perhaps, the issuance of an alarm.

The clinical care environment 800 includes not only the medical device 812 but also a computing system 824 and a central monitoring station 827. The central monitoring station 827 and the medical device 812 communicate with one another over the computing system 824, which includes the communications links 830a-830b. The central monitoring station 827 is at a remote location 833 relative to the local location 821. As used herein, in one sense, the term “remote” means a physically different location than where the medical device 812 is located. In one sense, “remote” may mean that the geographical location is physically different from the “local” location. In a second sense, “remote” also means that the location is outside the physical presence of the patient 806. Conversely, the medical device 812 is located remotely from the central monitoring station 827 in both of these senses, as well.

For example, patient 806 may be located in a room in which medical care is being delivered. The room may be in a medical care facility such as hospital, a hospice, an urgent care center, or a doctor's office, for example, depending on the implementation. The remote location may be in a different part of a municipality, or even in a different municipality or country than is the local location 821. The remote location 833 may be across town or down the street from the medial care facility in which the patient 806 is located. Or the “remote” location 833 may be in an office in the same building that the patient is being treated or down the hall from the room in which the patient 806 is located (i.e., the local location 821).

The computing system 824 may therefore be a combination of private and public networks. For example, the medical device 812 may communicate with the central monitoring station 827 over a medical facility's private network or over the Internet, which is a public network. Thus, communications between the medical device 812 and the central monitoring station 827 may occur in part or as a whole over private and/or public networks of the computing system 824. Accordingly, the communications links 830a-830b may be wired or wireless, depending on which is appropriate and/or desirable.

In the interest of completeness, selected aspects of the medical device 812 and the central monitoring station 827 will be presented. In general, it is contemplated by the present disclosure that medical device 812 and the central monitoring station 827, as well as any other computing device employed in the clinical care environment 800, includes electronic components, software, and/or electronic computing devices operable to receive, transmit, process, store, and/or manage data and information associated performing the functions of the system as described herein. This contemplation encompasses any suitable processing device adapted to perform computing tasks consistent with the execution of computer-readable instructions stored in a memory or a computer-readable recording medium.

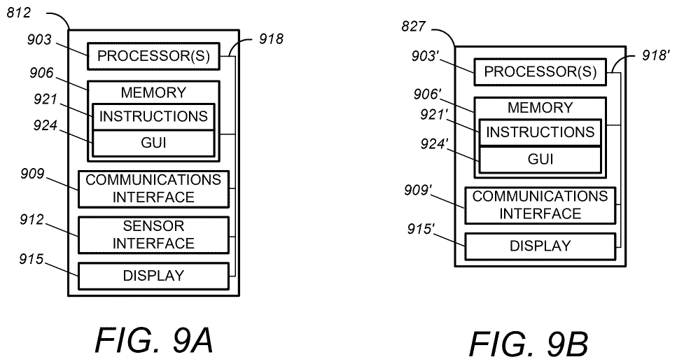

Referring now to FIG. 9A, the medical device 812 includes one or more processors 903, a memory 906, a communications interface 909, a sensor interface 912, a display 915, all communicating over a bus system 918. A set of instructions 921 and a graphical user interface (“GUI”) 924 reside on the memory 906. The medical device 812 receives the data acquired by the sensors 818 and leads 815, both shown in FIG. 8, through the sensor interface 912. The processors 903 execute the instructions 921 to process and analyze the acquired data in real time or near real time. The processors 903 may then display the processed data to a caregiver (not shown) at the patient's bedside on the display 915 using the GUI 924. The processors 903 also, through the execution of the instructions 921, transmit the data off of the device using the communications interface.

FIG. 9B depicts the central monitoring station 827. The central monitoring station 827 includes one or more processors 903′, a memory 906′, a communications interface 909′, and a display 915′, all of which communicate with one another over a bus system 918′. A set of instructions 921′ and a GUI 924′ reside on the memory 906′. Execution of the instructions 921′ by the processor(s) 918′ imparts the functionality of the central monitoring station 827, including interaction with the clinician 842, shown in FIG. 8, through the GUI 924′.

Those in the art having the benefit of this disclosure will appreciate that the patient monitor 812 and the central monitoring station 827 may, and probably will, include other components. These other components may implement common functionalities, like a power source. For instance, a power source (not shown) may include a self-contained power source such as a battery pack and/or include an interface to be powered through an electrical outlet, either directly or by way of a monitor mount. The power source may also be a rechargeable battery that can be detached allowing for replacement. In the case of a rechargeable battery, a small built-in back-up battery (or super capacitor) can be provided for continuous power to be provided during battery replacement.

Returning now to FIG. 8 and FIG. 9A-FIG. 9C collectively, the one or more processors 903, 903′ may be used for controlling the general operations of the respective device 812, 827. The one or more processors 903, 903′ may be any suitable processor-based resource. They may be, but are not limited to, a central processing unit (“CPU”), a hardware microprocessor, a multi-core processor, a single core processor, a field programmable gate array (“FPGA”), a controller, a microcontroller, an application specific integrated circuit (“ASIC”), a digital signal processor (“DSP”), or other similar processing device capable of executing any type of instructions, algorithms, or software for controlling the operation and performing the functions of respective device 812, 827. In some embodiments, the one or more processors 903, 903′ may comprise a processor chipset including, for example and without limitation, one or more co-processors.

The memories 906, 906′ may be single memory devices or one or more memory devices at one or more memory locations that may include, without limitation, one or more of a random-access memory (“RAM”), a memory buffer, a hard drive, a database, an erasable programmable read only memory (“EPROM”), an electrically erasable programmable read only memory (“EEPROM”), a read only memory (“ROM”), a flash memory, hard disk, various layers of memory hierarchy, or any other non-transitory computer readable medium. The memories 906, 906′ may be on-chip or off-chip depending on the implementation of the one or more processors 903, 903′.

The memories 906, 906′ may be used to store any type of instructions 921, 921′ associated with algorithms, processes, or operations for controlling the general functions and operations of the devices 812, 827. The instructions 921, 921′ may be any form of software, including, without limitation, firmware, executable applications, etc. Execution of the instructions 921, 921′ by the respective one or more processors 903, 903′ will impart the functionalities of the devices 812, 827 associated with the presently disclosed technique as discussed below.

The communications interfaces 909, 909′ may permit the respective network device 812, 827 to directly or indirectly (via, for example, a monitor mount) communicate with one or more computing networks and devices, workstations, consoles, computers, monitoring equipment, alert systems, and/or mobile devices (e.g., a mobile phone, tablet, or other hand-held display device). The communications interfaces 909, 909′ may include various network cards, interfaces, communication channels, cloud, antennas, and/or circuitry to permit wired and wireless communications with such computing networks and devices.

The communications interfaces 909, 909′ may be used to implement, for example, a BLUETOOTH® connection, a cellular network connection, and/or a WIFI® connection with such computing networks and devices. Example wireless communication connections implemented using the communication interfaces 909, 909′ include wireless connections that operate in accordance with, but are not limited to, IEEE802.11 protocol, a Radio Frequency For Consumer Electronics (“RF4CE”) protocol, and/or IEEE802.15.4 protocol (e.g., ZigBee® protocol). In essence, any wireless communication protocol may be used.

Additionally, the communications interfaces 909, 909′ may permit direct (i.e., device-to-device) communications (e.g., messaging, signal exchange, etc.) such as from, for example, a universal serial bus (“USB”) connection or other communication protocol interface. The communications interfaces 909, 909′ may also permit direct device-to-device connection to other devices such as to a tablet, computer, or similar electronic device; or to an external storage device or memory.

Those skilled in the art will appreciate that the implementation of the sensor interface 912 will turn strongly on the physiological parameters being monitored. Different kinds of data collected by different kinds of sensors will typically be conditioned differently and, so, the implementation of the sensor interface 912 will differ. However, although not required, most implementations of the sensor interface 912 will include analog-to-digital conversion of the data. Furthermore, as discussed elsewhere, not all medical devices will be patient monitors nor will they necessarily interface with sensors. Some medical devices therefore may omit a sensor interface.

The displays 915, 915′ may be used to present information to a caregiver or other user through the GUI 924, 924′ upon execution of the instructions 921, 921′ by the one or more processors 903, 903′. This may include, for example, the views shown in FIG. 4-FIG. 7. The displays 915, 915′ may be implemented using flat panel displays, cathode ray tube (“CRT”) displays, ultra-wide and curved displays, and/or any other suitable kind of display. Technologies may include, but are not limited to, liquid crystal display (“LCD”) such as in-plane switching (“IPS”) or vertical alignment (“VA”), light emitting diode (“LED”) such as organic light emitting diode (“OLED”), twisted-nematic panels, cathode ray tubes (“CRT”),

Note that the various components of devices 812, 827 may be implemented differently in any given embodiment. As a non-limiting example, in one embodiment the one or more processors 903 may be a single microprocessor while the one or more processors 903′ may be a processor chipset. Or the memory 906 may be implemented in a single RAM device while the memory 906″ may be implemented in a redundant array of independent disks. Those in the art having the benefit of this disclosure will appreciate still other examples of differences in implementation-specific differences across embodiments.

Returning to FIG. 8, the patient monitor 812 acquires data as described above and then processes and analyzes the data. As part of the processing and analyzing of the acquired data, the medical device 812 may detect an alarm condition. If so, an alarm is formulated and communicated. It is customary to announce the alarm using audio or visual cues. For example, the alarm may include, without limitation, a broadcast audio signal (e.g. a buzzing or beeping sound) and/or a visual signal (e.g., a flashing light). The alarm may also be communicated through the display 915. The alarm may also be transmitted to, for example, and without limitation, a centralized monitoring station (not shown), such as a nurses' station, over the computing system 824.

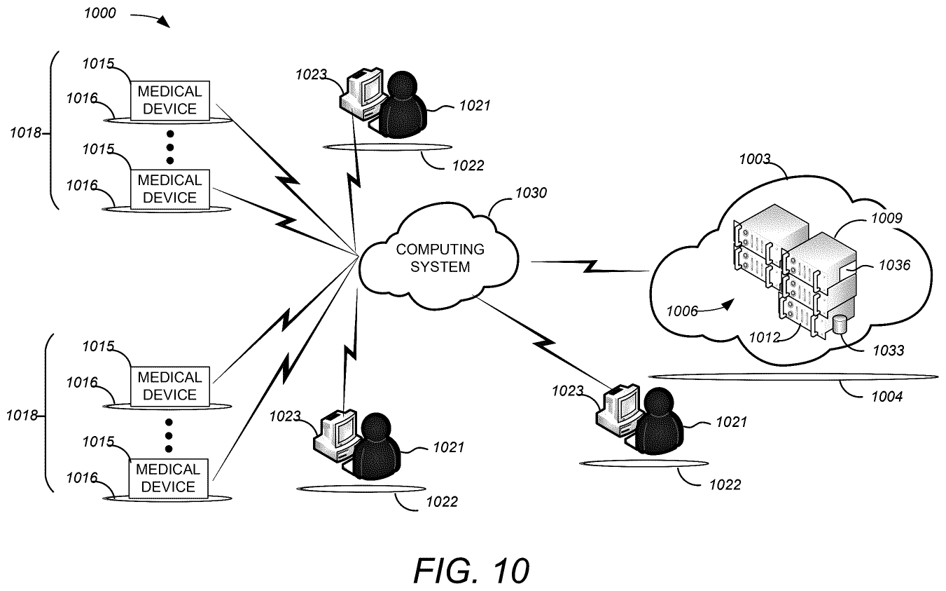

FIG. 10 depicts a clinical care environment 1000 in which a technological system may be implemented according to one or more examples. A cloud computing system 1003 at a location 1001 includes a plurality of computing resources 1003 that have been allocated to the implementation of the presently disclosed technique. The allocated resources may include, for example, process resources 1009 and memory or storage resources 1012. The clinical care environment 1000 further includes a plurality of medical devices 1015, each at a respective location 1016 and the medical devices grouped in clusters 1018. A plurality of clinicians 1021, each at a respective location 1022, also shown, each at a respective workstation 1023. The locations 1001, 1016, 1022 may be local or remote as discussed above. The computing devices (e.g., medical devices 1015, workstations 1023, cloud computing resources 1006) communicate with one another over the computing system 1030, also as discussed above.

Theoretically, there is no limitation on the number of medical devices 1015, workstations 1023 or locations 1016 subject only to the adequate availability of other computing resources. Such “other” computing resources may include, for example, bandwidth across the computing system 1030 and computing resources 1006 in the cloud computing system 1003. Accordingly, the number of medical devices 1015, workstations 1023, and computing resources 1006 are representative only and alternative embodiments may include more or fewer computing devices. Similarly, the number of locations 1016, 1022, and 1004 may vary by implementation. Although only one medical device 1015 is shown per location 1016, each location 1016 may host many medical devices 1015. Similarly, any number of workstations 1023 may be located in any given location 1022 and the cloud computing resources can be distributed across multiple locations 1004.

The medical devices 1015 operate in accordance with their programs to execute their programmed functionality relative to the patients (not shown in FIG. 10) to which they are assigned. In the course of these operations, alarms are detected and addressed by bedside caregivers or clinicians, neither of which are shown. In some cases, the clinicians 1021 may be monitoring the medical devices 1016 via the workstations 1023 and address the alarms. How an alarm may be addressed will depend on the condition that triggered the alarm. Addressing the alarm may be as complicated as delivering emergency treatment or as simple as muting the alarm.

Upon the occurrence of an alarm, the medical device 1015 transmits the alarm data as discussed above generated by the occurrence of the alarm. The alarm data may also include, in some embodiments, clinician feedback as to whether the alarm is a nuisance alarm. For example, if the clinician mutes the alarms quickly by addressing the underlying cause, it may be inferred that the alarm is a nuisance alarm. That information can then be included in the transmitted alarm data.

All such alarm data is transmitted over the computing system 1030 to the allocated computing resources 1006 of the cloud computing system 1003. The alarm data received over the computing system 1030 from all the medical devices 1015 is aggregated in the allocated storage resources 1012. The aggregated alarm data 1033 is then operated on by the allocated processing resources 1009, including both hardware and software resources 1036, as described above relative to FIG. 1. The hardware and software resources 1036 classify the alarms as nuisance or non-nuisance, prioritizes them, presents them to at least one clinician 1021. The hardware and software resources 1036 then receive the feedback of at least one of the clinicians 1021. The resultant set of actual nuisance alarms can then be used in one of several ways as described herein to help mitigate alarm fatigue.

Accordingly, the method(s) illustrated in FIGS. 1-3C may be deployed in a clinical care environment such as those shown in FIGS. 8-10. The method and apparatus of the present disclosure may therefore be implemented on a central monitoring station in cooperation with remote (e.g., bedside) medical devices such as patient monitors. The central monitoring station may follow one or more predetermined criteria that may include data from several clinical parameters to indicate a traffic lighting scheme for patients that need more attention than others. If all criteria are met, the patient will be indicated in the “red zone” demanding immediate attention. If a partial criterion is met, then the patient will be in the “yellow zone” and if no criterion is met, the patient will be in the “blue zone”. This color-coding scheme will help care givers/teletechs an opportunity to sort patients based on their clinical condition.

The presently disclosed method and apparatus therefore are integrated into and improve the functioning of the technological patient monitoring system in the clinical care environment. More particularly, the method and apparatus assist the caregivers in prioritizing automated alarms generated in the monitoring process and, thus, the physical response to such alarms. This, in turn, can help to mitigate “alarm fatigue”. Alarm fatigue is a complex and pervasive problem that occurs when clinicians are exposed to excessive numbers of alarms, which can result in the desensitization to alarm sounds and an increased rate of missed alarms. The presently disclosed method and apparatus thus increases the efficacy and efficiency of the patient monitoring process.

Accordingly, in a first embodiment, a computer-implemented method of prioritizing patient alarms is deployed in a patient monitoring system including a central monitoring station. The computer-implemented method comprises: receiving a patient alarm issued by the patient monitoring system when a monitored parameter deviates from a predetermined value; tracking a total number of patient alarms for a set of predefined parameters in a predefined time period; defining a plurality of priority levels for patient alarms, each priority level defined by a threshold number of patient alarms; and for each patient being monitored, displaying a visual indicator at the central monitoring station when the total number of patient alarms exceeds the threshold number of patient alarms for the set of predefined parameters in each of the plurality of priority levels, the visual indicator identifying the priority level reached by the monitored patient.

In a second embodiment, computer-implemented method of the first embodiment, the visual indicator comprises a patient specific indicator identifying the patient according to the priority level of the patient alarm.

In a third embodiment, the computer-implemented method of the second embodiment further comprises receiving a user input selecting the patient specific indicator and displaying a historical list of alarms that met Patient Alarm Prioritization criteria.

In a fourth embodiment, the computer-implemented method of the second embodiment further comprises receiving a user input selecting the patient specific indicator and transitioning the visual indicator from a blinking state to a static state.

In a fifth embodiment, the computer-implemented method of the second embodiment further comprises receiving a user input resetting the visual indicator.

In a sixth embodiment, the computer-implemented method of the first embodiment further comprises suggesting changes to alarm settings of the alarm is a frequently occurring alarm.

In a seventh embodiment, a central monitoring station for deployment in a clinical care environment comprises one or more processors, a display, and a memory. On the memory reside instructions that, when executed by the one or more processors, cause the one or more processors to perform a method. The method comprises: receiving a patient alarm issued by the patient monitoring system when a monitored parameter deviates from a predetermined value; tracking a total number of patient alarms for a set of predefined parameters in a predefined time period; defining a plurality of priority levels for patient alarms, each priority level defined by a threshold number of patient alarms; and for each patient being monitored, displaying a visual indicator on the display when the total number of patient alarms exceeds the threshold number of patient alarms for the set of predefined parameters in each of the plurality of priority levels, the visual indicator identifying the priority level reached by the monitored patient.

In an eighth embodiment, in the central monitoring station of the seventh embodiment, the visual indicator comprises a patient specific indicator identifying the patient according to the priority level of the patient alarm.

In a ninth embodiment, the central monitoring station of the eighth embodiment further comprises receiving a user input selecting the patient specific indicator and displaying a historical list of alarms that met Patient Alarm Prioritization criteria.

In a tenth embodiment, the central monitoring station of the eighth embodiment further comprises receiving a user input selecting the patient specific indicator and transitioning the visual indicator from a blinking state to a static state.

In the eleventh embodiment, the central monitoring station of the eighth embodiment further comprises receiving a user input resetting the visual indicator.

In a twelfth embodiment, the central monitoring station of the seventh embodiment further comprises suggesting changes to alarm settings of the alarm is a frequently occurring alarm.

In a thirteenth embodiment, in the central monitoring station of the seventh embodiment, the method further comprises receiving a plurality of patient alarms issued by the patient monitoring system from a plurality of medical devices, the received patient alarm being one of the plurality of patient alarms.

In a fourteenth embodiment, a patient monitoring system is deployed in a clinical care environment. The patient monitoring system comprises a plurality of medical devices and a central monitoring station. The medical devices monitor a respective physiological condition for each of a plurality of patients. The monitoring includes monitoring one or more physiological parameters and issuing one or more alarms when a monitored parameter deviates from a predetermined value. The central monitoring station performs a method comprising: receiving the patient alarms issued by the plurality of medical devices; tracking a total number of patient alarms for a set of predefined parameters in a predefined time period; defining a plurality of priority levels for the patient alarms, each priority level defined by a threshold number of patient alarms; and for each patient being monitored, displaying a visual indicator at the central monitoring station when the total number of patient alarms exceeds the threshold number of patient alarms for the set of predefined parameters in each of the plurality of priority levels, the visual indicator identifying the priority level reached by the monitored patient.

In a fifteenth embodiment, in the patient monitoring system of the fourteenth embodiment, the visual indicator comprises a patient specific indicator identifying the patient according to the priority level of the patient alarm.

In the sixteenth embodiment, the patient monitoring system of the fifteenth embodiment further comprises receiving a user input selecting the patient specific indicator and displaying a historical list of alarms that met Patient Alarm Prioritization criteria.

In a seventeenth embodiment, the patient monitoring system of the fifteenth embodiment further comprises receiving a user input selecting the patient specific indicator and transitioning the visual indicator from a blinking state to a static state.

In an eighteenth embodiment, the patient monitoring system of the fifteenth embodiment further comprises receiving a user input resetting the visual indicator.

In a nineteenth embodiment, the patient monitoring system of the fourteenth embodiment further comprises suggesting changes to alarm settings of the alarm is a frequently occurring alarm.

In a twentieth embodiment, the patient monitoring system of the fourteenth embodiment further comprises a computing system over which the medical devices and the central monitoring station communicate.

The detailed description is made with reference to the accompanying drawings and is provided to assist in a comprehensive understanding of various example embodiments of the present disclosure. Changes may be made in the function and arrangement of elements discussed without departing from the spirit and scope of the disclosure. Various embodiments may omit, substitute, or add various procedures or components as appropriate. For instance, features described with respect to certain embodiments may be combined in other embodiments. In addition, descriptions of well-known functions and constructions may be omitted for clarity and conciseness. Accordingly, those of ordinary skill in the art will recognize that various changes and modifications of the examples described herein can be made without departing from the spirit and scope of the present disclosure.

Use of the phrases “capable of,” “capable to,” “operable to,” or “configured to” in one or more embodiments, refers to some apparatus, logic, hardware, and/or element designed in such a way to enable the use of the apparatus, logic, hardware, and/or element in a specified manner. Use of the phrase “exceed” in one or more embodiments, indicates that a measured value could be higher than a pre-determined threshold (e.g., an upper threshold), or lower than a pre-determined threshold (e.g., a lower threshold). When a pre-determined threshold range (defined by an upper threshold and a lower threshold) is used, the use of the phrase “exceed” in one or more embodiments could also indicate a measured value is outside the pre-determined threshold range (e.g., higher than the upper threshold or lower than the lower threshold). The subject matter of the present disclosure is provided as examples of apparatus, systems, methods, circuits, and programs for performing the features described in the present disclosure. However, further features or variations are contemplated in addition to the features described above. It is contemplated that the implementation of the components and functions of the present disclosure can be done with any newly arising technology that may replace any of the above-implemented technologies.

Various modifications to the disclosure will be readily apparent to those skilled in the art, and the generic principles defined herein may be applied to other variations without departing from the spirit or scope of the present disclosure. Throughout the present disclosure the terms “example,” “examples,” or “exemplary” indicate examples or instances and do not imply or require any preference for the noted examples. Thus, the present disclosure is not to be limited to the examples and designs described herein but is to be accorded the widest scope consistent with the principles and novel features disclosed.

What is claimed is:

1. A computer-implemented method of prioritizing patient alarms in a patient monitoring system including a central monitoring station, the computer-implemented method comprising:
receiving one or more patient alarms issued by the patient monitoring system when a monitored parameter deviates from a predetermined value;
tracking a total number of patient alarms for a set of predefined parameters in a predefined time period;
defining a plurality of priority levels for patient alarms, each priority level defined by a threshold number of patient alarms; and
for each patient being monitored, displaying a visual indicator at the central monitoring station when the total number of patient alarms exceeds the threshold number of patient alarms for the set of predefined parameters in each of the plurality of priority levels, the visual indicator identifying the priority level reached by the monitored patient.

2. The computer-implemented method of claim 1, wherein the visual indicator comprises a patient specific indicator identifying the patient according to the priority level of the patient alarm.

3. The computer-implemented method of claim 2, further comprising:
receiving a user input selecting the patient specific indicator; and
displaying a historical list of alarms that met Patient Alarm Prioritization criteria.

4. The computer-implemented method of claim 2, further comprising:
receiving a user input selecting the patient specific indicator; and
transitioning the visual indicator from a blinking state to a static state.

5. The computer-implemented method of claim 2, further comprising receiving a user input resetting the visual indicator.

6. The computer-implemented method of claim 1, further comprising suggesting changes to alarm settings of the alarm is a frequently occurring alarm.

7. A central monitoring station for deployment in a clinical care environment, comprising:
one or more processors;
a display; and
a memory on which reside instructions that, when executed by the one or more processors, cause the one or more processors to perform a method comprising:
receiving a patient alarm issued by the patient monitoring system when a monitored parameter deviates from a predetermined value;
tracking a total number of patient alarms for a set of predefined parameters in a predefined time period;
defining a plurality of priority levels for patient alarms, each priority level defined by a threshold number of patient alarms; and
for each patient being monitored, displaying a visual indicator on the display when the total number of patient alarms exceeds the threshold number of patient alarms for the set of predefined parameters in each of the plurality of priority levels, the visual indicator identifying the priority level reached by the monitored patient.

8. The central monitoring station of claim 7, wherein the visual indicator comprises a patient specific indicator identifying the patient according to the priority level of the patient alarm.

9. The central monitoring station of claim 8, further comprising:
receiving a user input selecting the patient specific indicator; and
displaying a historical list of alarms that met Patient Alarm Prioritization criteria.

10. The central monitoring station of claim 8, further comprising:
receiving a user input selecting the patient specific indicator; and
transitioning the visual indicator from a blinking state to a static state.

11. The central monitoring station of claim 8, further comprising receiving a user input resetting the visual indicator.

12. The central monitoring station of claim 7, further comprising suggesting changes to alarm settings if the alarm is a frequently occurring alarm.

13. The central monitoring station of claim 7, wherein the method further comprises receiving a plurality of patient alarms issued by the patient monitoring system from a plurality of medical devices, the received patient alarm being one of the plurality of patient alarms.

14. A patient monitoring system deployed in a clinical care environment, the patient monitoring system comprising:
a plurality of medical devices monitoring a respective physiological condition for each of a plurality of patients, the monitoring including monitoring one or more physiological parameters and issuing one or more alarms when a monitored parameter deviates from a predetermined value; and
a central monitoring station performing a method comprising:
receiving the patient alarms issued by the plurality of medical devices;
tracking a total number of patient alarms for a set of predefined parameters in a predefined time period;
defining a plurality of priority levels for the patient alarms, each priority level defined by a threshold number of patient alarms; and
for each patient being monitored, displaying a visual indicator at the central monitoring station when the total number of patient alarms exceeds the threshold number of patient alarms for the set of predefined parameters in each of the plurality of priority levels, the visual indicator identifying the priority level reached by the monitored patient.

15. The patient monitoring system of claim 14, wherein the visual indicator comprises a patient specific indicator identifying the patient according to the priority level of the patient alarm.

16. The patient monitoring system of claim 15, further comprising:
receiving a user input selecting the patient specific indicator; and
displaying a historical list of alarms that met Patient Alarm Prioritization criteria.

17. The patient monitoring system of claim 15, further comprising:
receiving a user input selecting the patient specific indicator; and
transitioning the visual indicator from a blinking state to a static state.

18. The patient monitoring system of claim 15, further comprising receiving a user input resetting the visual indicator.

19. The patient monitoring system of claim 14, further comprising suggesting changes to alarm settings if the alarm is a frequently occurring alarm.

20. The patient monitoring system of claim 14, further comprising a computing system over which the medical devices and the central monitoring station communicate.
